(19) United States
(12) Patent Application Publication
(10) Pub. No.: US 2022/0188737 A1
(43) Pub. Date: Jun. 16, 2022

(54) SYSTEM FOR DETERMINING AN OPERATIONAL STATE OF AN AIRCREW ACCORDING TO AN ADAPTIVE TASK PLAN AND ASSOCIATED METHOD

(71) Applicant: DASSAULT AVIATION, PARIS (FR)

(72) Inventors: Lauren DARGENT, SAINT CLOUD (FR); Hervé GIROD, SAINT CLOUD (FR)

(21) Appl. No.: 17/550,283

(22) Filed: Dec. 14, 2021

(30) Foreign Application Priority Data
Dec. 15, 2020 (FR) FR 20 13237

(51) Int. Cl. G06Q 10/06; G16H 50/30

(52) U.S. Cl. CPC G06Q 10/063112; G06Q 10/063116; G16H 50/30

(57) ABSTRACT

A system for determining an operational state of an aircrew according to an adaptive task plan. The system comprising a computer adapted to determine for the or each crew member at least one theoretical baseline crew member cognitive state according to the task list assigned to the crew member, using a resource model. The computer is adapted to determine an indicator of fitness of the or each crew member to perform the task(s) of the task list assigned to the crew member, from an actual crew member cognitive state determined on the basis of physiological measurements on the crew member and measurements of the crew member's interaction with the aircraft, and from the theoretical baseline crew member cognitive states.

The present disclosure relates to a system for determining an operational state of the aircrew of a manned or unmanned aircraft based on an adaptive task plan.

The operational state of an aircraft includes the ability of a crew to perform its mission tasks based on its current state.

This system is intended in particular to evaluate in real time the operational state of the crew, with regard to all the tasks they have to carry out with the aircraft in the course of a mission. For example, the aircraft is manned and the crew is present in the cockpit of the aircraft. Alternatively, the aircraft is unmanned and the crew is in a UAV ground control station.

### BACKGROUND

An example of a mission is a civil aircraft mission, such as a commercial or private flight from a geographical point of departure to a geographical point of arrival, in which, during the mission phases, many tasks have to be performed in accordance with applicable procedures.

In one embodiment, the mission is a military aircraft (manned aircraft or UAV) mission, involving in particular reconnaissance or combat tasks, for example air-to-ground combat tasks to neutralise targets.

In such missions, the crew usually follows a task plan with a list of tasks to be performed, each associated with the time by which the task must be completed.

During a mission, the task plan is adaptive. This means that the list of tasks to be performed is updated automatically by the aircraft and validated by the crew, particularly in the light of events occurring during the mission. The crew can also decide on their own to change the task plan.

For example, mission plan or flight plan objectives may change, such as when a civil aircraft is diverted, or when a new target is to be handled for a military aircraft.

Similarly, the mission environment is likely to change, for example due to weather conditions in the civilian or military domain, or the emergence of additional threats in the military domain.

In some cases, the crew's resources, i.e. their ability to perform their tasks, are not adapted to the task plan.

This can be explained, for example, by the fact that a large number of additional tasks have to be carried out due to a breakdown or emergency on the aircraft. The crew may also be tired or stressed, which makes it difficult for them to carry out their duties.

Finally, in some critical cases, the aircrew may find themselves overwhelmed by all the tasks to be performed, or on the contrary, they may concentrate too much on one particular task, and neglect important tasks that should be done.

To check the condition of the crew, it is known to equip the aircraft cockpit with sensors measuring the crew's physiological condition. However, these sensors are only interested in the instantaneous state of a crew member, without linking this instantaneous state with the constraints that the crew undergoes to achieve the mission plan.

### SUMMARY

One aim of the present disclosure is to provide a system for monitoring crew behaviour during the execution of a task plan that reliably and dynamically determines the crew's ability to complete its mission.

To this end, a system of the aforementioned type is provided, comprising:
a module for determining crew tasks to be carried out, capable of defining, at each moment during a mission of the aircraft, a list of tasks to be carried out by at least one crew member as a function of updated mission objectives, and of an instantaneous mission context determined from measurements of the mission environment;
a crew resource determination module, adapted to determine for the or each crew member at least one theoretical baseline crew member cognitive state according to the task list assigned to the crew member, using a resource model;
a crew operational state determination module, adapted to determine an indicator of the fitness of the or each crew member to perform the task(s) of the task list assigned to the crew member, from an actual crew member cognitive state determined on the basis of physiological measurements on the crew member and measurements of the crew member's interaction with the aircraft, and from the theoretical baseline crew member cognitive state determined by the crew resource determination module.

The system according to the present disclosure may comprise one or more of the following features, taken alone or in any combination that is technically possible:
the operational state determination module is adapted to transition the fitness indicator between a state of fitness of the crew member to perform the task(s) of the task list assigned to the crew member and a state of unfitness of the crew member to perform the task(s) of the task list assigned to the crew member, based on a comparison between the actual crew member cognitive state and the theoretical crew member cognitive state;
it comprises a mission task reconfiguration module, suitable for modifying the task list to be performed by the crew member when the fitness indicator changes to the state of unfitness of the crew member to perform the task(s) of the task list assigned to the crew member.
the task reconfiguration module is suitable for deleting or/and postponing a task to be performed, performing a task instead of the crew member, deleting information given to the crew member, and/or replacing the information given to the crew member with other information.
it comprises a module for determining overall cognitive states of the crew member as a function of the physiological and interaction measurements of that crew member and a module for determining local cognitive states of the crew, adapted to determine local cognitive states related to the execution of tasks by the crew member as a function of the crew member's physiological measurements and aircraft interaction measurements, the crew operational state determination module being adapted to calculate the actual crew member cognitive state as a function of the local cognitive states and the overall cognitive states determined by the overall cognitive state determination module and the local cognitive state determination module, respectively, and to compare each local or overall crew member cognitive state with the theoretical baseline local or overall crew member cognitive state determined by the crew resource determination module based on the resource model;
the overall cognitive states are selected from a level of mental load, a level of engagement, a level of sleepiness, a level of hypoxia, and/or a level of stress of the crew member;
the local cognitive states are selected from a level of attention, a level of perseveration, a level of visual tunnelling, a level of auditory tunnelling in the performance of a task;
it comprises a module for measuring physiological states based on sensors for measuring physiological data, in particular based on sensors for measuring heart rate, pupil diameter, number of blinks, blood oxygenation, brain waves, evoked potentials, and based on a database of basic physiological states of the crew, the module for determining overall cognitive states determining the overall cognitive states from the physiological state measurements of the physiological state measurement module;
it comprises a module for measuring crew interaction states, based on sensors for measuring crew interaction states, in particular gaze position, use of physical commands, use of touch, and based on a model of expected interactions based on the list of tasks determined by the module for determining current crew tasks, the module for determining local cognitive states determining the local cognitive states from the interaction state measurements of the module for measuring interaction states;
the overall cognitive state determination module also determines the overall cognitive states from the interaction state measurements of the interaction state measurement module;
the local cognitive state determination module determines the local cognitive states also from the interaction state measurements of the interaction state measurement module;
the interaction states are selected from at least one level of attention, one level of performance, and one task completion strategy;
it comprises a context indication module, based on mission environment measurements, adapted to define at least one context indicator, the crew resource determination module being adapted to determine the theoretical baseline crew member cognitive state based on the or each context indicator;
the current crew task determination module is adapted to assign the list of tasks to the crew member according to a database of crew profiles determining the characteristics of each crew member and advantageously the task handling capabilities of the aircraft, and according to the mission objectives;
the crew resource determination module is adapted to assign, via the resource model, for the set of tasks to be performed, the overall cognitive state necessary to perform the tasks, advantageously according to the profile of the crew member given by the database and to the context indicator determination module;
the crew resource determination module is adapted to assign, via the resource model, for each task on the task list, the local cognitive state necessary to perform said task advantageously according to the profile of the crew member.

A method is also provided of determining an operational state of an aircrew of a manned or unmanned aircraft, based on an adaptive task plan comprising the following steps:
provision of a determination system as defined above;
definition, by the module for determining current crew tasks, at each moment during a mission of the aircraft, of a list of tasks to be carried out by at least one crew member as a function of updated mission objectives, and of an instantaneous mission context determined from environmental measurements of the mission;
determination, by the crew resource determination module, for the or each crew member, of at least one theoretical baseline crew member cognitive state based on the task list assigned to the crew member, using a resource model;
determination by the crew operational state determination module, of an indicator of the fitness of the or each crew member to perform the task(s) of the task list assigned to the crew member, from an actual crew member cognitive state determined on the basis of physiological measurements on the crew member and measurements of the crew member's interaction with the aircraft, and from the theoretical baseline crew member cognitive state determined by the crew resource determination module.

The method according to the present disclosure may comprise one or more of the following features, taken alone or in any combination that is technically possible:
the transition of the fitness indicator between a state of fitness of the crew member to perform the task(s) on the crew member's assigned task list and a state of unfitness of the crew member to perform the task(s) on the crew member's assigned task list based on a comparison between the actual crew member cognitive state and the theoretical crew member cognitive state;
it comprises a modification, by a mission task reconfiguration module, of the task list to be performed by the crew member when the fitness indicator changes to the state of unfitness of the crew member to perform the task(s) on the task list.

### BRIEF DESCRIPTION OF THE DRAWINGS

The present disclosure will be better understood upon reading the following description, given only as an example, and with reference to the attached drawings, in which:

FIG. 1 is a block diagram illustrating a first crew operational state determination system according to the present disclosure, integrated into an aircraft;

FIG. 2 is a block diagram illustrating the interactions between the different modules of the system of FIG. 1;

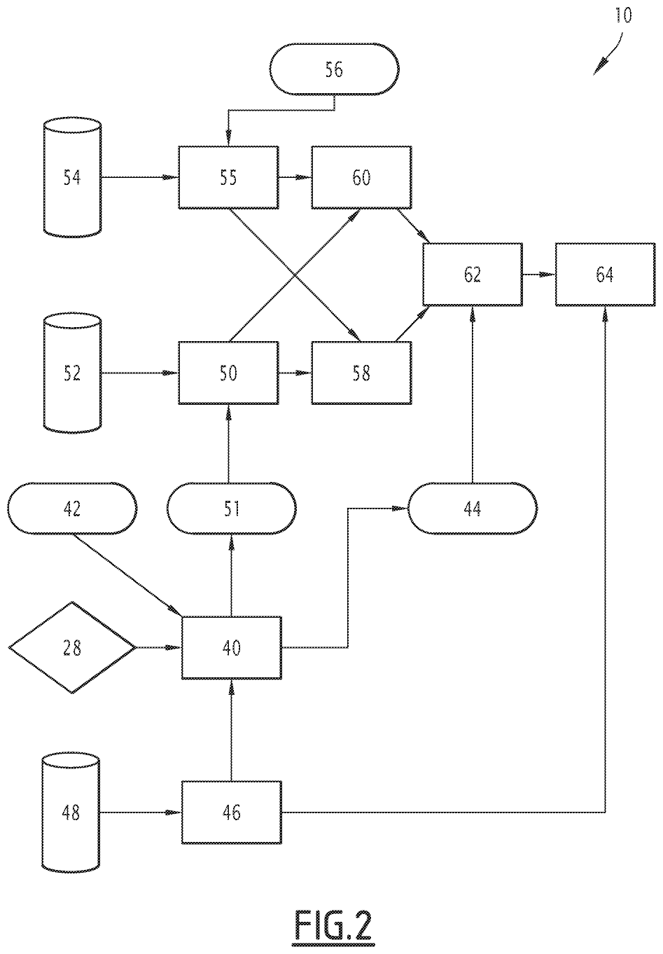

FIG. 3 is a flowchart illustrating a first example embodiment of a crew operational state determination method by means of the system of FIG. 1;

FIG. 4 is a flowchart illustrating a second example embodiment of a crew operational state determination method by means of the system of FIG. 1;

FIG. 5 is a flowchart illustrating a third example embodiment of a crew operational state determination method by means of the system of FIG. 1; and

FIG. 6 is a flowchart illustrating a fourth example embodiment of a crew operational state determination method by means of the system of FIG. 1.

FIG. 7 is a flowchart illustrating a fifth example embodiment of a crew operational state determination method by means of the system of FIG. 1.

### DETAILED DESCRIPTION

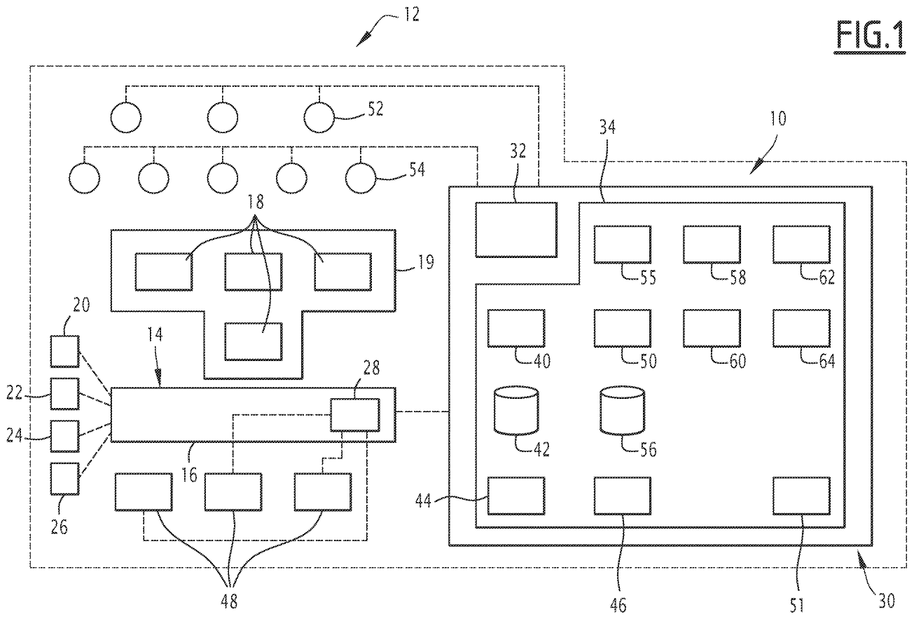

A first system 10 for determining the operational state of an aircrew of an aircraft 12, based on a task plan to be performed by the aircrew of the aircraft 12, is illustrated schematically in FIG. 1.

The system 10 is intended to be connected to or integrated into a central avionics system 14 comprising a central avionics unit 16 and at least one display unit 18 located in a control interface 19 of the aircraft 12.

The control interface 19 of the aircraft 12 is for example located in the aircraft 12 itself (in the cockpit), or in a remote control room of the aircraft 12 (in a ground station).

In particular, the central avionics unit 16 is connected to aircraft 12 equipment, which is intended to interact within the aircraft's functional systems.

The functional systems of the aircraft 12 include, for example, systems 20 for measuring the condition of the aircraft, systems 22 for external communication, and systems 24 for operating the aircraft controls. The functional systems of the aircraft 12 furthermore advantageously comprise weapons systems 26, in the case of a military aircraft.

The measurement systems 20 comprise, for example, components comprising sensors for measuring parameters external to the aircraft, such as temperature, pressure or speed, sensors for measuring parameters internal to the aircraft and its various functional systems and positioning sensors, such as GPS sensors, inertial units, and/or an altimeter.

The external communication systems 22 include, for example, components comprising radio, VOR/LOC, ADS, DME, ILS, radar systems, and/or satellite communication systems such as SATCOM.

The control systems 24 include components comprising actuators for operating aircraft controls, such as flaps, control surfaces, pumps, mechanical, electrical and/or hydraulic systems, and software actuators for configuring the avionics states of the aircraft.

Weapon systems 26, where present, advantageously comprise target tracking and weapon guidance components and weapons themselves, such as bombs or missiles.

The various systems 20 to 26 are connected to the central avionics unit 16, for example digitally, by at least one data bus running on an internal network within the aircraft 12.

The central avionics unit 16 comprises at least one task management system 28 capable of establishing, in real time, a list of crew tasks to be performed, which depends on mission objectives updated by measurements of the environment made by, for example, the measurement systems 20 or received by the communication systems 22.

The tasks to be performed are, for example, navigation tasks, to guide the aircraft 12 and move it according to a flight plan comprising at least geographical waypoints of the aircraft 12 during the mission and times when the waypoints are crossed.

Also, the tasks to be performed are, for example, communication tasks to be performed according to a communication plan comprising geographical communication points and/or times of passage at geographical communication points.

In addition, the tasks to be performed may be tasks related to events external to the aircraft, such as meteorology, including geographical waypoints or avoidance of weather phenomena, for example, in areas of turbulence and/or storms.

In the context of a military aircraft, the tasks to be performed advantageously further include reconnaissance tasks, air-to-ground or air-to-air combat preparation tasks, and air-to-ground or air-to-air combat tasks such as, for example, image analysis or weapon-firing from the aircraft.

The task list is usually determined initially, before the flight, from a mission planning system, based on mission objectives. Tasks are updated as the mission is executed, for example because new mission objectives are determined by the mission commander based on breakdowns and/or emergencies and/or external weather or tactical events.

The task management system 28 is then able to modify the task list, to add, delete, and/or change the order or characteristics of tasks to be performed by the crew, according to updated mission objectives and an updated mission context according to the environment around the aircraft 12. The task plan including the task list is thus adaptive. These changes are made within the limits of the system delegation provided by the crew and may be subject to crew agreement.

The crew operational state determination system 10 is schematically depicted in FIG. 1. In this example, the system 10 comprises a computer 30 comprising a processor 32 and a memory 34 that receives software modules that can be executed by the processor 32 to perform functions. Alternatively, the computer 30 comprises programmable logic components or dedicated integrated circuits to perform the functions of the modules described below.

With reference to FIG. 1, the memory 34 contains a module 40 for determining crew tasks to be performed at the current time, which is adapted to retrieve a list of tasks to be performed by the crew, according to updated mission objectives and/or the updated mission context, and to assign the tasks to the crew members, according to crew profile data contained in a database 42.

The memory 34 further contains a crew resource determination module 44 adapted to calculate the resources required by each crew member to perform each task on the task list assigned to that person and to determine a theoretical baseline operational state of each crew member to perform the tasks assigned to that person based on a resource model.

The memory 34 further contains a module 46 for determining at least one context indicator, suitable for defining at least one context indicator on the basis of mission context data derived from sensors 48 of the measurement systems 20 of the aircraft 12, or from data transmitted to the aircraft 12 via the communication systems 20.

The memory 34 further contains an interaction determination module 51, which is capable of determining the expected interactions with the aircraft systems, when the tasks on the task list are implemented by each crew member.

The memory 34 further contains a module 50 for measuring states of interactions with aircraft systems, based on measurements made by interaction measurement sensors 52 and expected interactions determined by the module 51, and a module 55 for measuring crew physiological states connected to sensors 54 for measuring physiological data of the aircrew of the aircraft 12 and to a database 56 of basic crew physiological data.

Finally, the memory 34 contains a module 58 for determining local cognitive states, suitable for evaluating at least one local crew cognitive state with respect to the execution of each task defined in the list of tasks to be carried out, and a module 60 for determining overall cognitive states of the crew related to the general physiological state of the crew with respect to all the tasks to be carried out.

A local crew cognitive state is a state of a mental process of a crew member associated with the performance of a particular task on the task list.

An overall crew cognitive state is a state of a crew member's mental process associated with the set of tasks to be performed by the crew member.

According to the present disclosure, the memory 34 further contains a crew operational state determination module 62, which is suitable for comparing at least one actual operational state of a crew member, in particular a overall cognitive state and/or a local cognitive state determined by the modules 58, 60, with a baseline theoretical operational state, in particular with a baseline theoretical overall cognitive state and/or a baseline theoretical local cognitive state, determined by the determination module 44 using the resource model.

The memory 34 further contains a task reconfiguration module 64 based on the operational state obtained by the state determination module 62, suitable for modifying the list of tasks to be performed by the crew member or the information provided to the crew.

The task determination module 40 is adapted to import, at any time, the tasks from the task management system 28 to obtain an ordered list of tasks, including for example target identification, flight plan monitoring, firing tasks to be performed, etc.

Each task is associated with at least one crew member, whose profile is obtained from the database 42. The crew profile includes, for example, a level of expertise of the crew member in performing the tasks on the task list, as well as preferred modes of interaction.

The preferred modes of interaction are, for example, possible alternative strategies for carrying out the tasks, in particular alternative task sequencing orders. For example, certain strategies for carrying out a task are known to an expert user with expertise in carrying out the task, enabling that person to use shortcuts or to optimise their interactions with the interfaces.

The resource determination module 44 is adapted, on the basis of the task lists, and on the basis of the resources required to implement the task list, obtained from a resource model, to determine, for each crew member, at least one baseline theoretical operational state of the crew member, in particular at least one baseline theoretical overall cognitive state and/or at least one baseline theoretical local cognitive state associated with each task for the crew member.

Examples of baseline overall cognitive states are an accepted theoretical level of mental load for the crew member, an accepted theoretical level of drowsiness for the crew member, an accepted theoretical level of crew member engagement for the crew member, an accepted theoretical level of hypoxia for the crew member (this accepted level being zero) and/or an accepted theoretical level of stress for the crew member.

The level of mental load is a numerical indicator of the demand on the crew member's mental capacity. The level of drowsiness is a numerical indicator of the degree of sleepiness of the crew member, especially in a state between wakefulness and sleep. The level of engagement is a numerical indicator of the crew member's overall attention to task completion. The level of hypoxia is a Boolean indicator of the adequacy or inadequacy of tissue oxygen requirements and oxygen supply for the crew member. The stress level is a numerical indicator of the nervous tension of the crew member.

These theoretical levels are predefined globally according to the crew profile and are intended to be compared with actual levels, determined from measured crew data, as described below. These levels are, for example, numerical levels measuring a value within a range of possible values, discrete levels measuring one of a plurality of possible discrete values, or Boolean levels having either an acceptable or an unacceptable value.

Examples of baseline local cognitive states for task performance are a minimum level of attention, a level of perseveration in performing each task, and a maximum level of visual and/or auditory tunnelling to perform each task in the task list.

The level of attention associated with a task is a numerical indicator that reflects the crew member's readiness to perform the task, in particular the ability of a crew to select and focus on specific information in a task. The level of perseveration is a numerical indicator that reflects, in a context that requires it, a lack of revision of a previously made decision in the performance of a task. The level of tunnelling is a numerical indicator of the allocation of the crew's attention to a specific sensory channel, to the detriment of others, which results in the crew's inability to take into account certain types of stimuli (e.g. sound and/or visual) related to the task at hand.

These levels are defined according to the crew member's crew profile and the crew member's assigned task list.

Theoretical baseline cognitive states are determined from the resource model which advantageously links each task to be performed to a time and/or resource level to be used by the crew member to perform the task.

The determination module 44 is thus adapted to assign for the set of tasks the necessary time as well as the necessary overall state (e.g. the necessary mental load) to perform the tasks according to the crew member profile given by the database 42 and the context indicators defined by the context indicator determination module 46. Similarly, it is proper to assign for each task in the task list the time needed and the local state needed (e.g. the level of attention needed) to perform the task according to the crew member's profile.

The context indicator determination module 46 is adapted to define a level of complexity and/or a level of danger, depending on measurements obtained by the environment sensors 48, in particular on the number of enemies present in the mission environment, the state of the weather, and/or the presence of dangerous areas.

The level of complexity and/or the level of danger is used by the determination module 44 to weight the resources needed to perform each task, in particular in terms of the time needed or the mental load needed to perform the tasks. For example, if the level of complexity or the level of danger increases, the time and mental load required to perform a task are likely to increase.

The interaction determination module 51 is adapted to determine the expected interactions with the aircraft systems by each crew member, and the expected performance to achieve these interactions, based on the list of tasks assigned to the crew member obtained from the determination module 40, the crew profile of the crew member as determined in the database 42 and the context indicators determined by the determination module 46.

Interactions with aircraft systems are, for example, screen areas or elements to be observed, keys or screen regions to be selected, or commands to be activated. The expected performance is, for example, a minimum speed for the sequence of actions to be carried out in order to implement one or more tasks, an error rate in the implementation of the tasks, or a total task completion rate among all the tasks to be carried out.

The interaction measurement sensors 52 are for example able to determine the position of the gaze on the cockpit 19 display units 18, the pressure and force of pressure on the controls, or the position of a control cursor of the aircraft systems on a display unit 18 or on another interface.

Based on the measurements made by the interaction measurement sensors 52, the interaction state measurement module 50 is adapted to compare the interactions with aircraft systems performed by the crew member and the interactions with aircraft systems expected for the crew member obtained from the module 51 based on the ordered list of tasks, to determine whether the crew member is performing each of the expected interactions.

The interaction state measurement module 50 is further adapted to determine whether the crew member meets the expected performance in terms of speed of execution, error rate and completion rate.

Based on the performance and actions carried out by the crew member, the interaction state measurement module 50 is adapted to determine a level of attention of the crew member to carry out each task, a level of performance of the crew member in carrying out each task, and a level of effectiveness of the strategy employed to manage the tasks according to the preferred modes of interaction defined above.

Physiological measurement sensors 54 include, for example, sensors for measuring heart rate, pupil diameter, blood oxygenation, emitted brain waves, posture (in particular via pressure mapping pads in the seat or/and via camera image analysis systems), sweating, or frontal oxygenation.

The physiological measurement sensors 54 may be partially shared with the interaction measurement sensors 52.

The physiological database 56, for each crew member, contains data on the basic physiological states of each crew member at rest.

These basic physiological state data are, for example, resting heart rate, resting pupil diameter, resting blood oxygenation rate, resting brain wave rate, resting posture, resting sweat rate, or resting frontal oxygenation rate.

On the basis of the measurements made by the physiological measurement sensors 54, compared with the basic physiological state data present in the database 56, the physiological state measurement module 55 is able to determine in particular a level of mental fatigue of the crew member, and/or a level of concentration of the crew member.

The level of mental fatigue is for example determined from a measured percentage of eye closure, a number and duration of blinks, a level of posture relaxation, a level of gesture repetition, and/or certain frequency spectral powers (e.g. alpha waves) predominating in the brain.

The level of concentration is for example determined from a prefrontal oxygenation level, e.g. a high prefrontal oxygenation level, a heart rate variability, e.g. a low heart rate variability, a variation in eye dispersion, e.g. a decrease in eye dispersion, and certain high brain frequency spectral powers (theta waves in the frontal area)

The local cognitive state determination module 58 is adapted to use the data produced by the physiological state measurement module 55 and the interaction state measurement module 50 to determine local cognitive states, related to the performance of each of the specific tasks that are provided in the ordered list of tasks.

As mentioned above, local cognitive states include, for example, a level of attention in performing tasks, for example, determined from analysis of the distribution of the driver's gaze directed toward their human/machine interfaces.

Local cognitive states comprise, for example, a level of perseveration. The level of perseveration is determined, for example, by frequency measurements of the brain, by a rate of reaction to stimuli, in particular a low rate of reaction to stimuli, or by a rate of fixation on the task, in particular a high rate of fixation on the task.

A high level of perseveration is attained, for example, when a pilot continues to attempt to land at all costs when external conditions would dictate a go-around.

Local cognitive states also preferably include a level of visual and/or auditory tunnelling, which reflects an excessive focus on a specific task, neglecting other tasks on the task list and/or visual and/or auditory stimuli external to the task. The level of tunnelling is determined, for example, from eye measurements, determination of lack of interaction on certain tasks, and determination of lack of response to certain stimuli (reflected, for example, by the amplitude of P300 evoked potentials in the brain, in particular by a low amplitude).

The overall cognitive state determination module 60 is adapted to use the data produced by the physiological state measurement module 55 and the interaction state measurement module 50 to determine overall cognitive states of each crew member.

Overall cognitive states include, for example, an overall level of mental load. This overall level of mental load is determined, for example, from prefrontal oxygenation, in particular high prefrontal oxygenation, heart rate variability, in particular a decrease in heart rate variability, performance on secondary tasks (delay, etc.), in particular a decrease in performance, variation in pupillary diameter, in particular an increase in pupillary diameter.

The overall cognitive states advantageously comprise an overall level of drowsiness. This overall level of drowsiness is determined for example from posture, in particular a relaxed posture, the number of eye blinks, in particular an increase in the number of eye blinks, and a determination of the absence of interactions.

The overall cognitive states advantageously include a level of engagement, which reflects the rate of completed tasks relative to the tasks to be completed. The level of engagement is determined for example from a ratio of brain frequency waves (beta/alpha+delta), also called “engagement index”, in particular higher than at rest, and/or from an analysis of eye data for example to identify a ratio “long fixations+short saccades/long saccades+short fixations”, in particular when this ratio is low.

The overall cognitive states advantageously include a level of hypoxia. The level of hypoxia is a Boolean indicator of the adequacy or inadequacy of tissue oxygen requirements and oxygen supply for the crew member. This level of hypoxia is a Boolean level. It is determined, for example, from a heart rate and respiratory rate above predetermined thresholds indicating abnormality, a drop in blood oxygen saturation below a threshold, incoherent sentences, and/or eye blinks above a predetermined threshold.

Overall cognitive states also preferably include a level of stress. The level of stress is determined for example from micro-sweating, in particular high micro-sweating, muscle tension, in particular a high muscle tension, heart rate variability, in particular a high heart rate variability, saccade/fixation ratio, in particular a high saccade/fixation ratio, voice frequency, in particular voice becoming more high-pitched, or/and sitting upright.

The crew operational state determination module 62 is adapted to analyse the overall cognitive states and the local cognitive states and to compare them respectively with the baseline overall cognitive states and the baseline local cognitive states calculated by the resource determination module 44 to determine a crew member fitness indicator, for example a Boolean indicator, suitable for transitioning between a state of fitness of the crew member to perform the tasks in the list of tasks assigned to the crew member and a state of unfitness of the crew member to perform tasks in the list of tasks assigned to the crew member.

For example, the crew operational state determination module 62 is adapted to evaluate whether the actual mental load level determined by the overall cognitive state determination module 60 is greater than the theoretical maximum mental load level calculated by the resource determination module 44. In such a case, it is able to change the crew member's fitness indicator from a state of fitness to a state of unfitness.

Similarly, the crew operational state determination module 62 is advantageously adapted to assess whether the actual stress level determined by the overall cognitive state determination module 60 is higher than the maximum stress level calculated by the resource determination module 44. In such a case, it is able to change the crew member's fitness indicator from a state of fitness to a state of unfitness.

Advantageously, the crew operational state determination module 62 is adapted to assess whether the actual level of drowsiness determined by the overall cognitive state determination module 60 is higher than the maximum level of drowsiness calculated by the resource determination module 44. In such a case, it is able to change the crew member's fitness indicator from a state of fitness to a state of unfitness.

Advantageously, the crew operational state determination module 62 is adapted to assess whether the actual level of engagement determined by the overall cognitive state determination module 60 is higher than the level of engagement calculated by the resource determination module 44. In such a case, it is able to change the crew member's fitness indicator from a state of fitness to a state of unfitness.

Advantageously, the crew operational state determination module 62 is adapted to evaluate whether the actual hypoxia level determined by the overall cognitive state determination module 60 is higher than the allowed hypoxia level, in this case a zero level. In such a case, it is able to change the crew member's fitness indicator from a state of fitness to a state of unfitness.

Furthermore, the crew operational state determination module 62 is advantageously adapted to evaluate whether the actual tunnelling level and/or the perseveration level determined by the local cognitive state determination module 58 exceeds the theoretical maximum tunnelling level and/or the theoretical maximum perseveration level determined by the resource determination module 44, respectively, and whether the attention level determined by the local cognitive state determination module 58 is lower than the attention level determined by the resource determination module 44.

In each of these cases, it is able to change the crew member's fitness indicator from a state of fitness to a state of unfitness.

The operational state determination module 62 is adapted, when a state of unfitness is determined, to define a type of task reconfiguration or crew information modification to be performed by the task configuration module 64.

The task configuration module 64 is advantageously able to modify the list of tasks in order to perform a task instead of the crew, to delete a task, or to postpone a task to be performed.

It is also adapted to generate an alert and/or an alarm in the event that the operational state determination module 62 determines the crew to be in a state of unfitness, or to temporarily suppress information displayed to the crew on the display units 18, and/or to display new information replacing existing information.

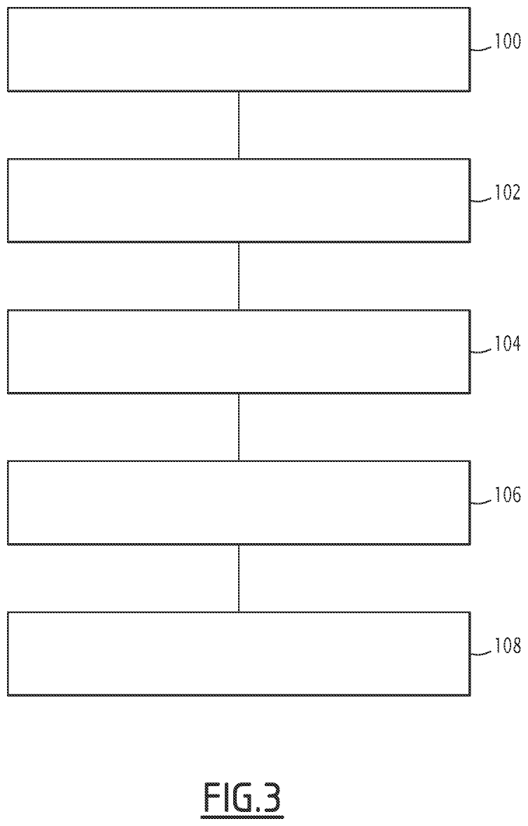

Examples of the implementation of a method for determining the operational state of a crew according to a task plan using the determination system 10 according to the present disclosure will now be described, with reference to FIGS. 3 to 7.

In the example shown in FIG. 3, in a civil aircraft context, the aircraft 12 is operating in a cruising phase, after the aircraft 12 has taken off.

The task management system 28 defines the tasks to be performed, which are essentially tasks of monitoring the aircraft's trajectory, in particular crossing waypoints, the fuel consumption of the aircraft 12 and communication tasks, when passing through various airspaces.

The monitoring crew member may enter a state of drowsiness.

As mentioned above, in step 100 of the method according to the present disclosure, the task determination module 40 establishes at each moment the list of tasks to be performed, and the distribution of the tasks between the crew members intended to perform them using the crew profile database 42.

Then, in step 102, the resource determination module 44 defines a maximum acceptable level of drowsiness for performing these tasks, based on, for example, a maximum level of drowsiness associated with each individual task.

In step 104, based on the measurements obtained from the physiological sensors 54, the physiological state measurement module 55 determines an increase in the frequency and duration of eye blinks, an increase in the percentage of eyelid closure, and a relaxed posture.

Based on these parameters, the overall cognitive state determination module 60 calculates an actual drowsiness level.

In step 106, the operational state determination module 62 determines that the actual drowsiness level is greater than the maximum allowable drowsiness level for the current task set. It therefore changes the crew member's fitness indicator from the state of fitness of the crew member to perform the tasks in their list of assigned tasks to the state of unfitness of the crew member to perform the tasks in their list of assigned tasks.

The operational state determination module 62 also determines that the type of reconfiguration is to trigger a wake-up alarm and temporarily automate the piloting of the aircraft.

In step 108, the reconfiguration module 64 therefore sends instructions to the aircraft system to generate an alarm in the cockpit 19.

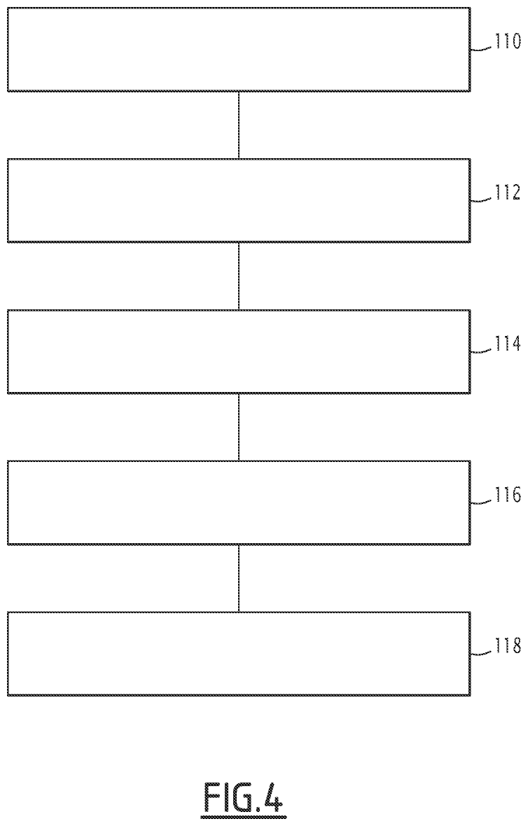

In a second example, illustrated in FIG. 4, during an approach phase to a landing strip with a strong crosswind, the pilot is focusing on a flying task.

If an important alarm occurs, it might not be given priority.

To that end, the task determination module 40 determines a list of tasks related to flying, and a list of tasks related to alarm handling, which may be a red CAS alarm requiring priority action by the pilot from the list of tasks.

In step 110, the interaction modelling module 51 determines that the crew member should handle the tasks associated with the alarm, by performing a number of interactions in the cockpit 19 including on the display units 18 and/or using the controls.

In step 112, the interaction state measurement module 50 determines from the cursor position and the gaze position that the crew member is not performing any of the interactions related to the alarm.

In addition, the interaction state measurement module 50 establishes that the crew member is not responding to audio and visual stimuli resulting from the alarm that are outside that person's field of vision.

On this basis, it determines a level of attention of the crew member to perform each task, and a level of performance of the crew member in performing each task.

Similarly, in step 114, the physiological state measurement module 55 determines a pupil diameter with little visual dispersion, a low heart rate variability, e.g. 20% less than the resting heart rate variability, and a high heart rate relative to the resting heart rate, e.g. at least 20% more than the resting heart rate, from databases 56 of basic physiological data.

The physiological state measurement module 55 furthermore determines an absence of amplitude of the P300 evoked potentials following a sound stimulus (alarm).

Based on the information obtained by the modules 50, 55, the overall cognitive state determination module 60 determines a mental load level and a stress level. The local cognitive state determination module 58 determines a level of tunnelling.

In step 116, the operational state determination module 62 then determines that the actual mental load level is greater than the maximum mental load level determined by the determination module 44 and that the tunnelling level exceeds the maximum tunnelling level determined by the determination module 44. It defines a state of unfitness of the crew member to perform the required tasks, in particular to handle mission alarms. The crew member is here in a state of inattentional deafness.

The operational state determination module 62 further determines that the action to be taken would be to temporarily suppress the display of information on which the pilot is focused.

In step 118, the reconfiguration module 64 therefore drives the display units 18 in the cockpit 19 to remove at least one piece of information relating to crosswind flying, to enable the pilot to identify that another urgent alarm is present and needs to be addressed.

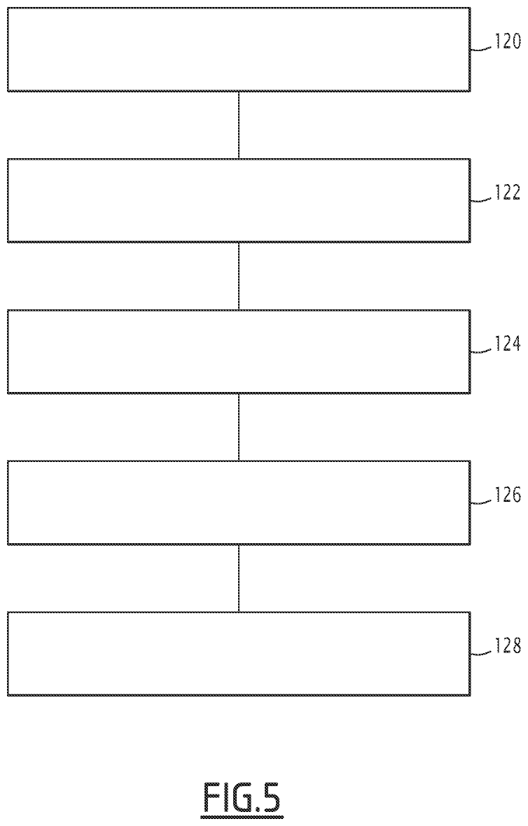

In a third example, shown in FIG. 5, the system 10 detects a state of panic present in a crew member.

This can happen, for example, when multiple alarms are displayed.

As before, in step 120, the task determination module 40 determines that a plurality of tasks are to be performed simultaneously and defines for each task a mental load level and a stress level associated with the task.

The interaction modelling module 51 determines the expected interactions with the cockpit interface and the expected performance to perform the monitoring tasks.

In step 122, based on the measurements of the interaction measurement sensors 52, the interaction state measurement module 50 compares the expected interactions for each crew member with the aircraft systems obtained from the module 51 based on the ordered list of tasks. It identifies a dispersion in interactions, and the fact that no task is being completed.

It therefore calculates an attention level and an engagement level. These levels are low.

Furthermore, in step 124, based on the physiological sensors 54, the physiological state measurement module 55 determines the presence of numerous saccades in the movement of the eyes, a very high heart rate, and sudden heavy sweating.

On this basis, the overall cognitive state determination module 60 calculates a stress level. This level of stress is high.

In step 126, the operational state determination module 62 then identifies a high stress level, greater than the maximum stress level set by the determination module 44. This level of stress is characteristic of a state of panic that renders the crew member unfit to perform safety-critical tasks.

It identifies a level of attention and a level of engagement below the minimum level of attention and the minimum level of engagement, respectively, determined by the determination module 44.

It therefore defines a state of unfitness of the crew member to perform the required tasks.

The operational state determination module 62 further determines that a summary of the information and procedure to be followed is to be performed by the reconfiguration module 64.

In step 128, the reconfiguration module orders the display units 18 of the central avionics unit 16 to provide a verbal and graphical summary of the information and procedure to be followed.

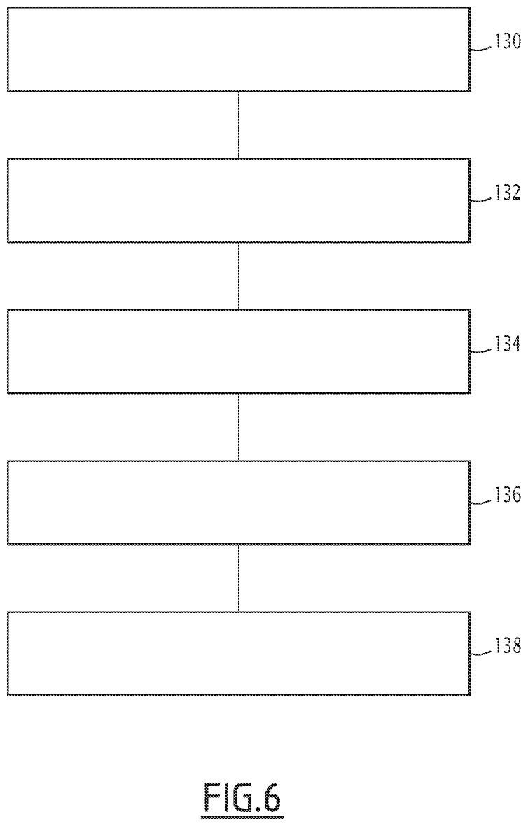

Another example will now be described with reference to FIG. 6, in the context of a military aircraft in high-altitude air combat.

As before, in step 130, the task determination module 40 determines the list of tasks to be performed in this air combat phase. The interaction determination module 51 determines the expected interactions with the aircraft systems by the crew member, and the expected performance to achieve these interactions, based on the list of tasks assigned to the crew member obtained from the determination module 40, the crew profile of the crew member as determined in the database 42 and the context indicators determined by the determination module 46.

In step 132, based on the data from the interaction sensors 52, the interaction state measurement module determines that the reaction time has increased significantly, and that the responses do not match what is expected for the interactions.

The overall cognitive state determination module 60 thus determines a level of engagement, and the local cognitive state determination module 58 determines a level of attention. These levels are low.

In parallel, in step 134, based on the data from the physiological sensors 54, the physiological state measurement module 55 determines a decrease in blood oxygen saturation, and an abnormal posture characterised by an absence of movement.

The overall cognitive state determination module 60 deduces therefrom a level of hypoxia. This level of hypoxia is unacceptable.

In step 136, the operational state determination module 62 identifies that the hypoxia level is greater than a maximum hypoxia level determined by the determination module 44, in this case a zero level. It therefore defines a state of unfitness of the crew member to perform the required tasks.

It also determines the actions to be taken by the reconfiguration module 64, which are to allow the central avionics unit 16 to take over flying, and to perform an emergency descent.

In step 138, the reconfiguration module 64 sends commands to the central avionics unit 16 to perform this emergency take-over and descent.

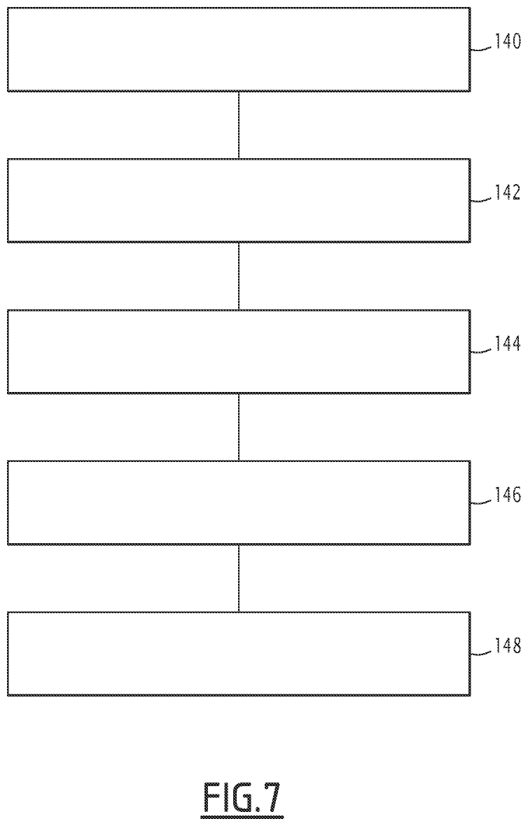

In another example, shown in FIG. 7, again in the context of a military aircraft, during an air-to-ground combat mission, the pilot is required to perform many tasks in a limited time.

In step 140, the task determination module 40 determines the list of tasks to be performed. The interaction determination module 51 determines the expected interactions with the aircraft systems by the crew member, and the expected performance to achieve these interactions, based on the list of tasks assigned to the crew member obtained from the determination module 40, the crew profile of the crew member as determined in the database 42 and the context indicators determined by the determination module 46.

In step 142, based on the data from the interaction sensors 52, the interaction state measurement module 50 determines repetitive errors in the interactions to be performed and a decrease in performance, in particular for tasks that are not prioritised or not scheduled in the initial task list.

The overall cognitive state determination module 60 thus determines a level of engagement, and the local cognitive state determination module 58 determines a level of attention. These levels are very high.

In parallel, in step 144, based on the data from the physiological sensors 54, the physiological state measurement module 55 determines a high prefrontal oxygenation, above a given threshold, the dominance of theta and beta waves at the frontal level, a large pupil diameter, a very low heart rate variability. From this, it deduces a high level of concentration.

The overall cognitive state determination module 60 calculates a mental load level on this basis. This level of mental load is high.

In step 146, the operational state determination module 62 then determines a mental load level greater than the maximum mental load level determined by the determination module 44. It therefore defines a state of unfitness of the crew member to carry out all the tasks required.

The operational state determination module 62 then determines a type of reconfiguration whereby the central avionics unit 16 automatically takes over certain lower-priority tasks.

In step 148, the reconfiguration module 64 therefore sends instructions to the central avionics unit 16 to take over the tasks thus defined.

The determination system 10 according to the present disclosure is thus able to establish an operational state of the crew, according to a resource model built on the list of crew tasks, by separately measuring the physiological states and interaction states of the crew, even if these measurements presumably come from data derived from the same sensors, and by separately determining local cognitive states, related to the performance of one task, and overall cognitive states, related to the tasks as a whole.

The determination system 10 updates the operational state as the task list changes, for example if a new task is added, or as the mission objectives change, or as the context observed in the environment changes.

This makes the determination system 10 very interactive, as it dynamically takes into account the context of the mission, and the resources of the crew.

Thanks to its generic functional architecture, the determination system 10 is also easily parameterised to adapt to different types of sensors, missions and platforms. It takes into account the specific characteristics of the crew members.

Advantageously, the determination system 10 further establishes reconfiguration actions to help solve the identified problems.

Thus, the determination system 10 easily interprets the crew's state, which improves the crew's performance and reduces their stress and mental load.

As indicated above, the local cognitive state determination module 58 is adapted to use the data produced by the physiological state measurement module 55 and the interaction state measurement module 50 to determine local cognitive states, related to the performance of each of the specific tasks that are provided in the ordered list of tasks.

The overall cognitive state determination module 60 is adapted to use the data produced by the physiological state measurement module 55 and the interaction state measurement module 50 to determine overall cognitive states of each crew member.

Thus, the same physiological measurement sensor 54, or the same interaction measurement sensor 52, can be used to determine at least one local cognitive state referring to a task and at least one overall cognitive state.

For example, when the sensor 52, 54 is an eye tracker, observing the parameters of eye opening percentage and blink rate can advantageously identify the level of drowsiness of the crew member, which is an overall physiological state. In addition, a sharply decreasing eye opening percentage may also indicate a state of drowsiness.

The area of gaze fixation, associated with the level of eye saccades, makes it possible, for example, to identify a level of attention, advantageously defined by the rate of attentional fixation on a given task, given that a task can be associated with a set of attentional areas in the cockpit. This characterises a local physiological state.

In the case of a sensor 52, 54 for establishing an electroencephalogram, the observation of brain waves establishes overall physiological states (drowsiness, mental load).

The observation of responses (measured electrical brain potential) to auditory stimuli within the brain (N100, P300) establishes local physiological states (e.g. a level of attention to an alarm or to information).

For the determination of the actual cognitive states, the determination modules 58, 60 are adapted to implement machine learning algorithms or non-learning algorithms.

Machine learning algorithms can be neural networks and/or decision trees. Non-learning algorithms are for example expert rules.

The training of the algorithms is advantageously performed as follows.

The measurement data from the sensors 52, 54 are recorded over experimental phases carried out by a number of crew members allowing supervised learning.

The training consists of several steps including a labelling of the measurement data to establish local or overall cognitive states.

This labelling can be done manually by external observers who have witnessed the experimental phases, and validated by the crew members concerned or manually, after the fact, by reviewing a video recording of the experimental phase with the crew member concerned.

Alternatively, the labelling can be carried out automatically, based on the results of subjective tests given to the crew members during the experimental phases (e.g. NASA-TLX test for mental workload) or automatically, based on numerical results of the crew members' performance during the data recording (score, reaction time, etc.).

Alternatively, labelling is achieved by a mixture of all these techniques.

The labelled data are then anonymised, normalised by the baseline of each crew member to avoid inter-individual variability, and advantageously mixed and separated into several sets for the training, fine-tuning and testing of algorithms.

Then, machine learning algorithms are trained on this data, or the business rules of the non-learning algorithms are established.

In one example, it is first possible to consider an existing fleet of aircraft and to make measurements of sensors made anonymous on all the pilots of the fleet, then to correlate the measurements on the ground after the flights with the observed states (following pilot questionnaires, and/or labelling of experts), and then to integrate these results into the determination system 10. This can be carried out iteratively.

Once this is done, during a flight, the determination module 60 is adapted to establish at each instant actual overall cognitive states of the crew member as a function of the physiological and interaction measurements of the crew member and the determination module 58 is adapted to establish at each instant actual local cognitive states relating to the execution of tasks by the crew member as a function of the physiological and interaction measurements of the crew member, by implementing the aforementioned algorithms.

Likewise, as described above, the crew resource determination module 44 is adapted to determine for the or each crew member at least one theoretical baseline crew member cognitive state according to the task list assigned to the crew member, using a resource model.

These baseline cognitive states are advantageously overall baseline cognitive states (e.g. level of mental load, level of drowsiness) or local baseline cognitive states (e.g. prioritisation of attention to task A over task B).

Each overall or local baseline cognitive state determined using the resource model by the module 44 corresponds to an overall or local actual cognitive state determined by a determination module 58, 60.

Thus, during flight, the determination module 62 compares each overall or local actual cognitive state determined by a determination module 58, 60 with a corresponding overall or local baseline cognitive state determined using the resource model by the module 44 to obtain the fitness indicator, as described above.

The resource model is suitable for determining at least one overall cognitive baseline state (e.g. mental workload required) based on the current task(s) on the task list, based on the crew member profile given by the database 42, and possibly based on context indicators defined by the context indicator determination module 46.

Similarly, the resource model is suitable for determining at least one local cognitive baseline state (e.g. the level of attention required) based on at least one task from the task list, based on the crew member's profile, and possibly based on a defined prioritisation of tasks.

The resource model advantageously implements machine learning algorithms or non-learning algorithms.

Machine learning algorithms can be neural networks and/or decision trees. Non-learning algorithms are for example expert rules.

The training of the algorithms is advantageously carried out on created data sets or by expertise.

The training of the algorithms is advantageously carried out as described above, by implementing experimental phases.

The baseline cognitive states are for example predefined by expertise. As a variant, baseline cognitive states are labelled from missions which have been carried out correctly, for which the operator reacted in adequacy with the tasks carried out (for example with a subjective feedback from the operator that he did not feel any problem with the tasks carried out).

Once the resource model has been trained or tuned, it can be used by providing it with an input of a list of tasks to be performed, a profile of the crew member given by the database 42, and possibly context indicators defined by the context indicator determination module 46.

The resource model then provides as output at least one baseline cognitive state, notably a overall baseline cognitive state corresponding to all the tasks given as input (for example: level of mental load, level of drowsiness) and/or a local baseline cognitive state corresponding to at least one task of the list of tasks provided as input (for example: prioritisation of attention to a task A over a task B).

The crew operational state determination module 62 is in such a case adapted to determine an indicator of the fitness of the or each crew member to perform the task(s) of the task list assigned to the crew member, based on an actual crew member cognitive state determined on the basis of physiological measurements on the crew member and measurements of the crew member's interaction with the aircraft 12, and from the theoretical baseline crew member cognitive state determined by the crew resource determination module.

In a first illustrative example, during a transit for a long flight, the resource model indicates that a high level of mental load (above a predefined threshold) is not normal, while a level of drowsiness above a predefined threshold is acceptable.

Based on the physiological and interaction measurements, the determination module 60 determines that the pilot has a normal load and is drowsy. The determination module 62 then determines that the crew member fitness indicator is in the state of fitness of the crew member to perform the tasks on the task list.

On the contrary, during a landing with inclement weather, the resource model indicates that a high level of mental load (above a predefined threshold) is acceptable, while a level of drowsiness above a predefined threshold is not acceptable.

Based on the physiological and interaction measurements, the determination module 60 determines that the pilot has a normal load and is drowsy. The determination module 62 then determines that the crew member fitness indicator is in the state of unfitness of the crew member to perform the tasks on the task list.

In another illustrative example, the resource model indicates that the pilot's mental load level (corresponding to the overall baseline state) should not exceed a predefined maximum baseline level, and that the pilot's attention (corresponding to a local baseline state) should be focused on task A as a priority.

Based on the physiological and interaction measurements, the determination module 60 establishes the mental load level of the pilot, and the determination module 62 determines that this actual mental load level is well below the value of the predefined maximum baseline level established by the resource model.

On the other hand, still based on the physiological and interaction measurements, the determination module 60 establishes that the pilot's attention is focused on a task B with a lower priority than task A according to the resource model.

The determination module 62 then determines that the crew member fitness indicator is in the state of unfitness of the crew member to perform the tasks on the task list.

What is claimed is:

1. A manned or unmanned aircraft aircrew operational state determination system, according to an adaptive task plan, the system comprising a computer,
the computer being configured to define, at each moment during a mission of the aircraft, a list of tasks to be carried out by at least one crew member as a function of updated mission objectives, and of an instantaneous mission context determined from measurements of a mission environment;
the computer being configured to determine for the at least one crew member at least one baseline theoretical crew member cognitive state according to the task list assigned to the at least one crew member, using a resource model;
the computer being configured to determine a fitness indicator of the at least one crew member to perform the task(s) of the task list assigned to the at least one crew member, from an actual crew member cognitive state determined on the basis of physiological measurements on the at least one crew member and of measurements of the at least one crew member's interaction with the aircraft, and from the baseline theoretical crew member cognitive state determined by the computer.

2. The system according to claim 1, wherein the computer is configured to transition the fitness indicator between a state of fitness of the at least one crew member to perform the task(s) of the task list assigned to the at least one crew member and a state of unfitness of the at least one crew member to perform the task(s) of the task list assigned to the at least one crew member, based on a comparison between the actual crew member cognitive state and the baseline theoretical crew member cognitive state.

3. The system according to claim 2, wherein the computer is configured to modify the task list to be performed by the at least one crew member when the fitness indicator changes to the state of unfitness of the at least one crew member to perform the task(s) of the task list assigned to the at least one crew member.

4. The system according to claim 3, wherein the computer is configured to delete or/and postpone a task to be performed, to perform a task instead of the at least one crew member, to delete information given to the at least one crew member, and/or to replace the information given to the at least one crew member with other information.

5. The system according to claim 1, wherein the computer is configured to determine overall cognitive states of the at least one crew member as a function of the physiological and interaction measurements of the at least one crew member and is configured to determine local cognitive states related to the execution of tasks by the at least one crew member as a function of the at least one crew member's physiological measurements and aircraft interaction measurements, the computer being configured to calculate the actual crew member cognitive state as a function of the local cognitive states and of the overall cognitive states determined by the computer, and to compare each local or overall crew member cognitive state with a baseline theoretical local or overall crew member cognitive state determined by the computer based on the resource model.

6. The system according to claim 5, wherein the overall cognitive states are selected from a level of mental load, a level of engagement, a level of sleepiness, a level of hypoxia, and/or a level of stress of the at least one crew member.

7. The system according to claim 5, wherein the local cognitive states are selected from a level of attention, a level of perseveration, a level of visual tunnelling, or a level of auditory tunnelling in the performance of a task.

8. The system according to claim 5, wherein the computer is configured to obtain physiological states measurements based on physiological data measuring sensors and based on a database of basic physiological states of the crew, the computer being configured to determine the overall cognitive states from the physiological state measurements.

9. The system according to claim 8, wherein the physiological data measuring sensors are sensors for measuring heart rate, pupil diameter, number of blinks, blood oxygenation, brain waves, and/or evoked potentials.

10. The system according to claim 1, wherein the computer is configured to obtain crew interaction states measurements, from crew interaction states sensors, and from a model of expected interactions based on the list of tasks determined by the computer, the computer being configured to determine local cognitive states from the interaction state measurements.

11. The system according to claim 10, wherein the crew interaction states sensors comprise gaze position sensors, physical commands use sensors, and touch sensors.

12. The system according to claim 10, wherein the crew interaction states are selected from at least one level of attention, one level of performance, and one task completion strategy.

13. The system according to claim 1, wherein the computer is configured, based on mission environment measurements, to define at least one context indicator, and to determine the baseline theoretical crew member cognitive state based on the at least one context indicator.

14. The system according to claim 1, wherein the computer is configured to assign the list of tasks to the at least one crew member according to a database of crew profiles determining the characteristics of each crew member, and/or the task handling capabilities of the aircraft, and according to the mission objectives.

15. A method of determining an operational state of an aircrew of a manned or unmanned aircraft, based on an adaptive task plan comprising:
providing the determination system according to claim 1;
defining, via the computer, at each moment during a mission of the aircraft, a list of tasks to be carried out by at least one crew member as a function of updated mission objectives, and of an instantaneous mission context determined from environmental measurements of the mission;
determining, via the computer, for the at least one crew member, at least one baseline theoretical crew member cognitive state based on the task list assigned to the at least one crew member, using a resource model;
determining, via the computer, an indicator of the fitness of the at least one crew member to perform the task(s) of the task list assigned to the at least one crew member, from an actual crew member cognitive state determined on the basis of physiological measurements on the at least one crew member and measurements of the at least one crew member's interaction with the aircraft, and from the baseline theoretical crew member cognitive state determined by the computer.

16. The method according to claim 15, further comprising transitioning, via the computer, the fitness indicator between a state of fitness of the at least one crew member to perform the task(s) on the at least one crew member's assigned task list and a state of unfitness of the at least one crew member to perform the task(s) on the at least one crew member's assigned task list based on a comparison between the actual crew member cognitive state and the theoretical crew member cognitive state.

17. The method according to claim 16, further comprising modifying, via the computer, the task list to be performed by the at least one crew member when the fitness indicator changes to the state of unfitness of the at least one crew member to perform the task(s) on the task list.
